(19) United States
(12) Patent Application Publication
(10) Pub. No.: US 2026/0020835 A1
(43) Pub. Date: Jan. 22, 2026

(54) Analysis System for Evaluating Physiological Tissue State

(71) Applicant: National Taiwan University, Taipei City (TW)

(72) Inventors: Pei-Hsing Chen, Taipei City (TW); Tai-Horng Young, Taipei City (TW); Jin-Shing Chen, Taipei City (TW); Po-Jen Shih, Taipei City (TW)

(21) Appl. No.: 18/954,362

(22) Filed: Nov. 20, 2024

(30) Foreign Application Priority Data
Jul. 17, 2024 (TW) 113126730

(51) Int. Cl. A61B 8/08; A61B 8/00

(52) U.S. Cl. CPC A61B 8/08; A61B 8/4236; A61B 8/5223; A61B 8/54

(57) ABSTRACT

An analysis system for evaluating physiological tissue states comprises a wave emitting unit designed to generate a detection wave for a physiological tissue; a wave receiving unit configured to receive a feedback wave reflected through the physiological tissue as a transmission medium; a control unit, controlling the wave emitting unit to output the detection wave at predetermined time intervals and correspondingly recording the feedback wave received by the wave receiving unit; and an analysis unit, allowing the control unit to control the wave emitting unit to dynamically adjust the detection wave output by the wave emitting unit based on the stored multiple audio signals, and analyzes the recorded feedback wave against stored comparison parameter to obtain a characteristic parameter, thereby enabling the establishment of a characteristic model for the physiological tissue.

### CROSS-REFERENCE TO RELATED APPLICATIONS

This application claims the priority of Republic of China Patent Application No. 113126730 filed on Jul. 17, 2024, in the State Intellectual Property Office of the R.O.C., the disclosure of which is incorporated herein by reference.

### BACKGROUND OF THE INVENTION

### Field of the Invention

The invention relates to an analysis technology for detecting the state of physiological tissue. More specifically, it pertains to an analysis system that utilizes audio transmission to evaluate the state of physiological tissue.

### Descriptions of the Related Art

When a patient visits a medical facility, a stethoscope provides the physician with the most immediate and convenient examination tool. Conditions such as pulmonary edema, pulmonary fibrosis, airway narrowing, lung obstruction, and bronchitis can be diagnosed more efficiently than through image interpretation. Respiratory waves can provide sufficient information even before imaging results are confirmed, without waiting for the lungs to develop substantial lesions. However, with the advancement of diagnostic tools such as ultrasound, computed tomography scans (CT scans), and X-rays, the use of stethoscopes has gradually declined, especially among younger physicians who no longer dedicate effort to learning auscultation, leading to the clinical near-extinction of auscultation skills.

Today, many experts recommend replacing traditional stethoscopes with ultrasound, CT scans, X-rays, and end-tidal CO2 monitoring. However, these diagnostic tools still face challenges such as large equipment size, accessibility, time consumption for obtaining results, labor-intensive equipment cleaning and disinfection, risk of pathogen transmission during patient transport, patient safety concerns, increased healthcare costs, and the risk of infection for healthcare team members.

Moreover, ultrasound diagnostic devices are not suitable for examining organs like the lungs. Due to the varying density of organ tissues, ultrasound conduction differs. If the tissue being examined is covered by air or bone, ultrasound cannot penetrate, limiting its effectiveness in detecting deep-seated lesions in the body.

In light of this, the technical problem to be solved by this invention is how to design a diagnostic tool that is not limited by the presence of air, bone, blood, or moisture surrounding the body's tissues, yet can still effectively detect the condition of these tissues. Additionally, the diagnostic tool should allow for automated interpretation and ease of use.

### SUMMARY OF THE INVENTION

In view of the aforementioned shortcomings of prior art, the present invention provides an analysis system for evaluating physiological tissue state, which comprises: a wave emitting unit installed on a first fixing member, and the first fixing member is attached to the surface of the physiological tissue to be state-evaluated, and the wave emitting unit is used to generate a detection wave for the physiological tissue; a wave receiving unit is installed on a second fixing member, and the second fixing member is set at a predetermined distance from the first fixing member and attached to the surface of the physiological tissue, and the wave receiving unit is used to receive a feedback wave, and the feedback wave is the reflection of the detection wave transmitted through the physiological tissue as the transmission medium; a control unit, electrically connected to the wave emitting unit and the wave receiving unit, used to control the wave emitting unit to output the detection wave at a predetermined time interval and correspondingly record the feedback wave received by the wave receiving unit, and an analysis unit, electrically connected to the control unit, used to store multiple audio signals and comparison parameters, enabling the control unit to control the wave emitting unit to dynamically adjust the detection wave output by the wave emitting unit based on the multiple audio signals, and the analysis unit processes the feedback wave recorded by the control unit and the comparison parameters to obtain a characteristic parameter, thereby establishing a characteristic model for the physiological tissue.

In one embodiment of the above-described analysis system for evaluating physiological tissue state, the control unit selects an audio signal from the multiple audio signals to control the wave emitting unit to output the detection wave based on the selected audio signal. The control unit provides the feedback wave received by the wave receiving unit to the analysis unit, allowing the analysis unit analyzes the waveform amplitude of the feedback wave against the comparison parameter to determine whether the state of the physiological tissue is pathological or normal, and the analysis unit then sets the waveform determined to be in the pathological state as the characteristic parameter and stores the characteristic parameter in the characteristic model.

In one embodiment of the above-described analysis system for evaluating physiological tissue state, when the analysis unit analyzes the waveform amplitude of the feedback wave against the comparison parameter, the control unit further selects multiple audio signals from the multiple audio signals to control the wave emitting unit to output the detection wave based on the selected audio signals. The control unit provides the feedback wave received by the wave receiving unit to the analysis unit, allowing the analysis unit processes the time-domain information of the feedback wave using Fourier transform to obtain frequency domain information of the feedback wave. The analysis unit analyzes the frequency peaks in the frequency domain information against the comparison parameter to determine whether the state of the physiological tissue is in the pathological state or the normal state, and the analysis unit then sets the waveform determined to be in the pathological state as the characteristic parameter and stores the characteristic parameter in the characteristic model.

In one embodiment of the above-described analysis system for evaluating physiological tissue state, the control unit further comprises a setting module that allows the user to set the desired state analysis mode. The analysis mode comprises: analyzing the amplitude of the feedback wave against the comparison parameter, analyzing the frequency peaks in the frequency domain information of the feedback wave against the comparison parameter, or simultaneously analyzing both the waveform amplitude of the feedback wave and the frequency peaks in the frequency domain information of the feedback wave.

Therefore, the analysis system for evaluating physiological tissue state of the present invention uses waves as the detection transmission means for the physiological tissue to be evaluated, effectively addressing the limitations of existing ultrasound inspection instruments in detecting organs such as the lungs, where air, moisture, or bones act as poor conductors, thereby hindering effective detection and interpretation. Furthermore, in the analysis system for evaluating physiological tissue state of the present invention, the first fixing member and the second fixing member are fixedly attached to the skin, for example, the chest, allowing the wave emitting unit mounted on the first fixing member to actively emit detection waves toward the lungs. The wave receiving device, oriented towards the lungs, receives the feedback wave reflected after the detection wave passing through the lungs, and the analysis unit processes the feedback wave to obtain the sensing results, thereby enabling to automatically detect and provide objective values without the need for interpretation by professional medical personnel and to provide scheduled or continuous monitoring detection functions, achieving real-time monitoring and prevention effects. Moreover, the analysis system for evaluating physiological tissue state of the present invention provides multiple continuous wave signals for detection, enabling the distinction of the severity of the condition. Unlike existing ultrasound inspection instruments, which use a single frequency signal and produce image data with only two forms of representation (either highlighted or fully lit), the present invention enhances the readability of diagnostic results compared to existing ultrasound inspection instruments.

### BRIEF DESCRIPTION OF THE DRAWINGS

FIG. 1 is a schematic diagram of the component arrangement of an embodiment of the analysis system for evaluating physiological tissue state according to the present invention.

FIG. 2A is a schematic diagram of waveform amplitude changes of the received feedback waves under analysis of the normal thorax versus pneumothorax in the analysis system for evaluating physiological tissue state according to the present invention.

FIG. 2B is a schematic diagram of waveform amplitude changes of the received feedback waves under analysis of the normal thorax versus pleural effusion in the analysis system for evaluating physiological tissue state according to the present invention.

FIG. 3A is a schematic diagram of the characteristic frequency waveforms of normal left and right lungs, obtained by applying Fourier transform to the time-domain information of the received feedback waves to obtain frequency domain information in the analysis system for evaluating physiological tissue state according to the present invention.

FIG. 3B is a schematic diagram of the characteristic frequency waveforms of a normal side versus a pleural effusion side of the left and right lungs, obtained by applying Fourier transform to the time-domain information of the received feedback waves to obtain frequency domain information in the analysis system for evaluating physiological tissue state according to the present invention.

FIG. 3C is a schematic diagram of the characteristic frequency waveforms of a normal side versus a pneumothorax side of the left and right lungs, obtained by applying Fourier transform to the time-domain information of the received feedback waves to obtain frequency domain information in the analysis system for evaluating physiological tissue state according to the present invention.

### DETAILED DESCRIPTION OF THE PREFERRED EMBODIMENT

Embodiments of the present invention will now be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art.

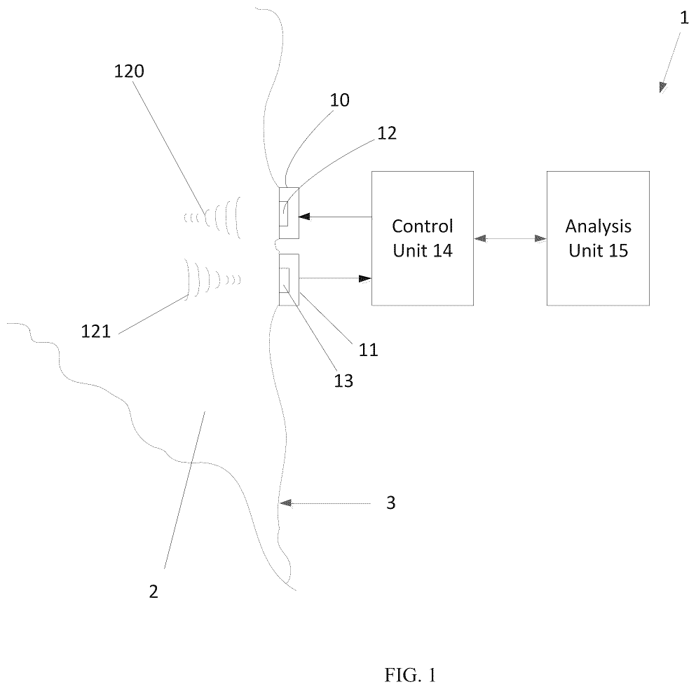

Firstly, refer to FIG. 1, which is a schematic diagram of the component arrangement of an embodiment of the analysis system for evaluating physiological tissue state of the present invention. This embodiment of the analysis system for evaluating physiological tissue state 1 comprises: a first fixing member 10, a second fixing member 11, a wave emitting unit 12, a wave receiving unit 13, a control unit 14, and an analysis unit 15. The wave emitting unit 12 is installed on the first fixing member 10. The first fixing member 10 is attached to the covering surface of a physiological tissue to be state-evaluated, and the aforementioned covering surface is the skin 3 of the human body. In the following embodiment, the lung 2 is used as the physiological tissue to be state-evaluated for example, but it is not limited to this; the physiological tissue could also be the stomach, liver, bladder, uterus, thyroid, breast, or intestines. The wave emitting unit 12 is used to produce a detection wave 120 to the lung 2. Moreover, the wave comprises a sound wave, but is not limited to it.

The wave receiving unit 13 is installed on the second fixing member 11, and the second fixing member 11 is set and attached to the skin 3 covering the lung 2 with a predetermined interval from the first fixing member 10. The wave receiving unit 13 is used to receive a feedback wave 121, and the feedback wave 121 is the reflection of the detection wave 120 transmitted through the lung 2 as the transmission medium. The first fixing member 10 and the second fixing member 11 are patches similar to commercially available electrotherapy patches or electrode patches. During the evaluation operation, the first fixing member 10 and the second fixing member 11 are attached to the skin 3 covering the lung 2 in a manner similar to how they would penetrate the lung 2, being positioned one in front of the other.

The control unit 14 is electrically connected to the wave emitting unit 12 and the wave receiving unit 13, controlling the wave emitting unit 12 to output the detection wave 120 at predetermined time intervals and correspondingly recording the feedback wave 121 received by the wave receiving unit 13.

The analysis unit 15 is electrically connected to the control unit 14 and can be connected via wired or wireless means. The analysis unit 15 performs data calculation functions for the control unit 14. The analysis unit 15 can be a microprocessor electrically connected to the control unit 14; or the analysis unit 15 can be a single device like a mobile phone or an external device, and the single device can be electrically connected to the control unit 14 wirelessly. The analysis unit 15 stores multiple audio signals and comparison parameters, enabling the control unit 14 to control the wave emitting unit 12 to dynamically adjust the detection wave 120 output by the wave emitting unit 12 based on the multiple audio signals. The analysis unit 15 analyzes the feedback wave 121 recorded by the control unit 14 against the comparison parameter to obtain a characteristic parameter and establish a characteristic model for the physiological tissue.

The analysis unit 15 can analyze the feedback wave 121 in the following two ways: (1) analyzing the waveform amplitude of the feedback wave 121; and (2) analyzing the frequency peaks in the frequency domain information of the feedback wave 121. These methods are described as follows:

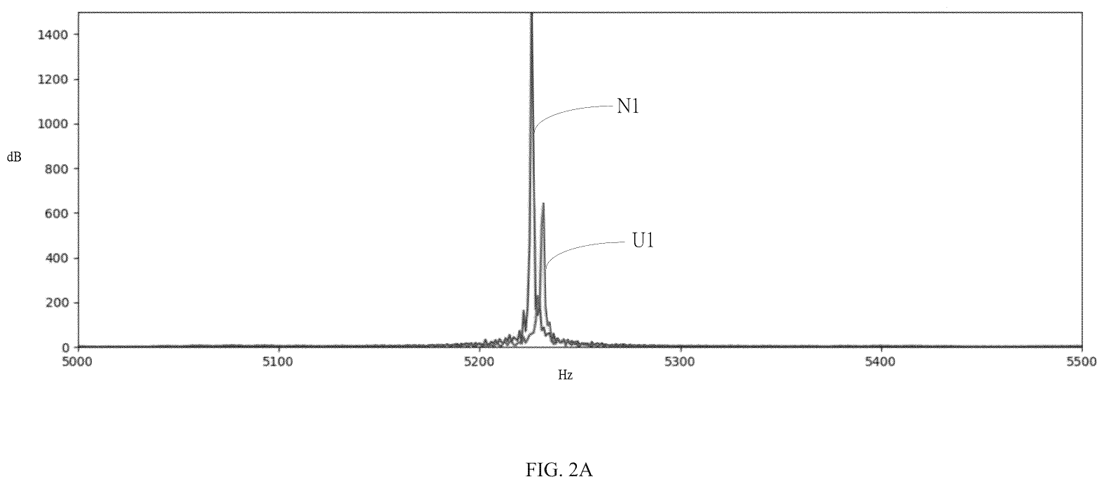

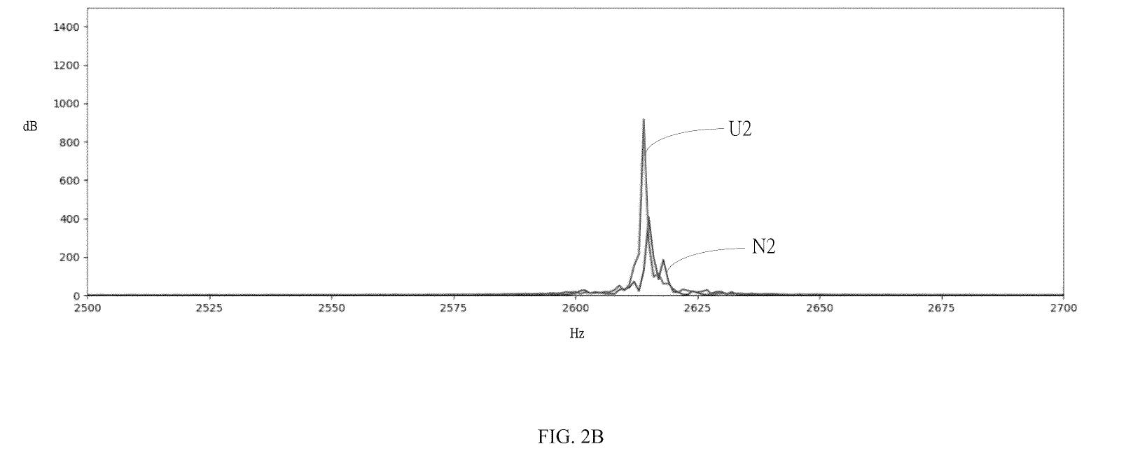

Firstly, regarding the analysis of the waveform amplitude of the feedback wave 121, the control unit 14 selects an audio signal from the multiple audio signals stored in the analysis unit 15 to control the wave emitting unit 12 to output the detection wave 120 based on the selected audio signal to the lung 2. The control unit 14 provides the feedback wave 121 received by the wave receiving unit 13 to the analysis unit 15, allowing the analysis unit 15 to analyze the waveform amplitude of the feedback wave 121 against the comparison parameter to determine whether the lung 2 is in a pathological state or a normal state and to set the waveform determined to be in the pathological state as the characteristic parameter and store the characteristic parameter in the characteristic model. It should be noted that, in the analysis operation of waveform amplitude, a specific audio signal such as 2000 Hz is provided during outputting detection signal such as in the range of 200 Hz to 800 Hz, because it is prone to causing mutual interference among the received feedback signals, during outputting the detection signal; therefore, a specific audio frequency signal is provided to facilitate analysis and simplify the analyzed feedback signal. For example, the analysis system for evaluating physiological tissue state of the present invention evaluates the degree of pneumothorax or pleural effusion in the chest cavity by using the chest cavity as the transmission medium for the detection wave and assessing the feedback wave to assist in determining the severity of the disease. Under typical conditions, the speed of wave from a wave source in air is 330 m/s, while in water, it is 1480 m/s. The frequency of wave does not change within the same medium; the frequency is determined solely by the source of the wave. However, the speed and wavelength of the wave will change when it passes through different media. Therefore, when a wave passes through a patient with pleural effusion, the medium being water, the wavelength and speed will differ compared to when the wave passes through a patient with pneumothorax. This principle allows the assessment of whether the patient has pneumothorax or pleural effusion, as well as the severity of the condition. In the methods of practical operation, the analysis system for evaluating physiological tissue state 1 of the present invention controls the wave emitting unit 12 via the control unit 14 to output the detection wave 120, for example, transmitting waves in the 2000˜3000 Hz range. The analysis unit 15 is used to evaluate the changes in amplitude and frequency of the feedback wave 121 received by the wave receiving unit 13. It can be observed that in areas with water or high degree of solidification, the amplitude decay of the feedback wave 121 received by the wave receiving unit 13 will vary under fixed frequency and transmission amplitude conditions. In the case of a normal lung, the situation will resemble air conduction with a large amplitude decay, as shown in FIG. 2A, where the vertical axis represents wave intensity and the horizontal axis represents the receiving frequency. Waveform U1 represents the condition of pneumothorax, and waveform N1 represents the condition of a normal chest cavity, where pneumothorax leads to reduced amplitude. However, if there is water or solidification, the amplitude decay will be smaller, as shown in FIG. 2B, where waveform U2 represents the condition of pleural effusion, and waveform N2 represents the condition of a normal chest cavity, where pleural effusion leads to increased amplitude. By analyzing the amplitude decay, the character of the object within the chest cavity can be assessed, thereby providing an objective, data-driven method that can replace traditional imaging examinations.

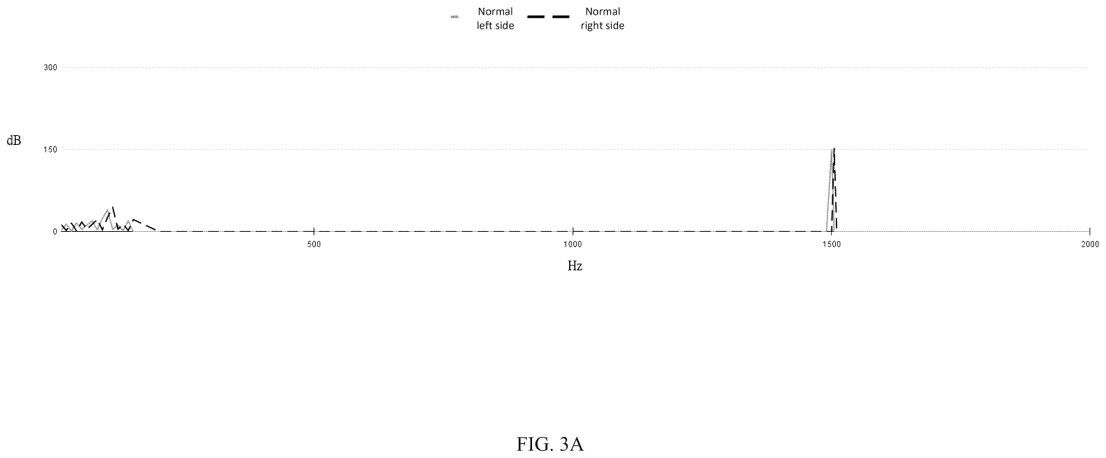

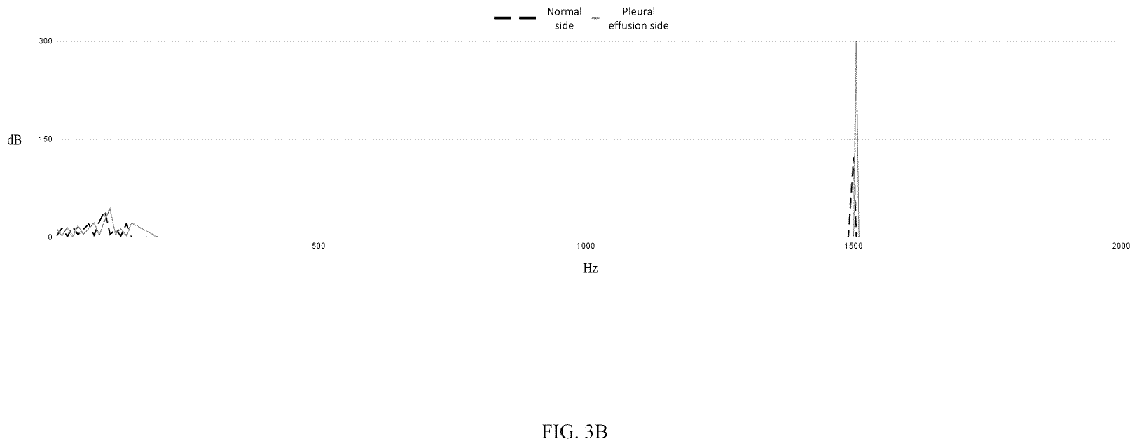

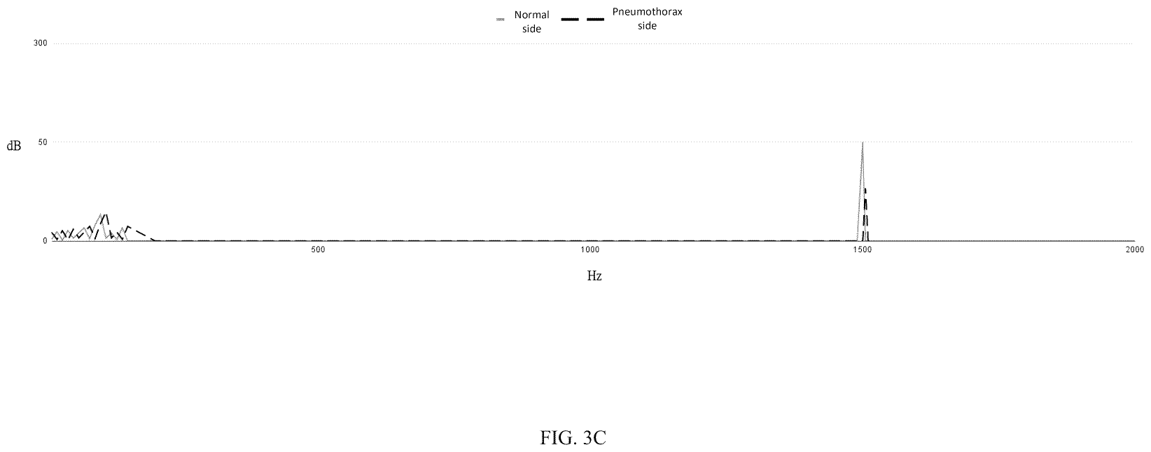

Furthermore, in terms of analyzing the frequency peaks in the frequency domain information of the feedback wave 121, the control unit 14 controls the wave emitting unit 12 to output the detection wave based on multiple wave frequency signals stored in the analysis unit 15. The control unit 14 then provides the feedback wave 121 received by the wave receiving unit 13 to the analysis unit 15, where the analysis unit 15 processes the time-domain information of the feedback wave 121 using Fourier transform to obtain the frequency domain information of the feedback wave 121. The frequency peaks in this frequency domain information are analyzed against the comparison parameter to determine whether the lung 2 is in the pathological state or the normal state, and the analysis unit 15 then sets the waveform determined to be in the pathological state as the characteristic parameter and stores the characteristic parameter in the characteristic model. Specifically, the waveform of the multiple wave frequency signals stored in the analysis unit 15 is a sine wave Y(t)=Asin (αt), wherein Y(t) is the wave intensity at time t, and α is a control variable used to adjust the frequency of the wave frequency signal. The control unit 14 selects multiple wave frequency signals from the multiple wave frequency signals to control the wave emitting unit 12 to output according to the selected multiple wave frequency signals. The wave frequency signals penetrate the skin 3 and reach the lung 2, producing different signal waveforms depending on the resistance and texture of the chest cavity and lungs during transmission. The wave receiving unit 13, placed on the chest cavity being tested, can collect the current signal waveform of the subject's chest cavity and lung in the time domain. When the wave emitting unit 12 outputs detection waves 120 with multiple wave frequency signals, using the chest cavity and lung as the transmission medium, the multiple wave frequency signals consist of a continuous frequency band, for example, wave frequencies increasing from 100 Hz, 120 Hz, 140 Hz, 160 Hz, 180 Hz, or such as from 200 Hz, 220 Hz, 240 Hz, 260 Hz, 280 Hz, etc. Additionally, the frequency band could also decrease, such as from 200 Hz, 180 Hz, 160 Hz, 140 Hz, 120 Hz, or such as from 300 Hz, 280 Hz, 260 Hz, 240 Hz, 220 Hz, etc. That is, the detection is carried out using a wave frequency segment composed of multiple wave frequencies. The wave receiving unit 13 receives the feedback wave 121 and provides the feedback wave 121 to the analysis unit 15 via the control unit 14 for analysis. The analysis unit 15 processes the time-domain information of the feedback wave 121 using Fourier transform to obtain the frequency domain information of the feedback wave 121, which is then compared with comparison parameter to determine the relationship between characteristic frequencies and diseases, as shown in FIGS. 3A to 3C. The FIG. 3A shows the characteristic frequencies under normal left and right lung conditions. The FIG. 3B shows the characteristic frequencies when one side of the lung is normal and the other side has pleural effusion. The FIG. 3C shows the characteristic frequencies when one side of the lung is normal and the other side has pneumothorax. The unchanging frequency peak, analyzed from the multiple and continuous increasing or decreasing detection waves, represents the characteristic frequency. As can be seen, the analysis system for evaluating physiological tissue state 1 of the present invention is not limited by the medium of the physiological tissue being tested under wave detection. Therefore, this invention can also be applied to estimate the degree of vascular sclerosis in dialysis, liver cirrhosis, vascular sclerosis in stroke patients, and quantifying the degree of renal solidification, thus potentially replacing ultrasound examination instruments that require a high degree of subjective judgment. Additionally, the present invention is more miniaturized in volume compared to ultrasound examination instruments, making it very convenient to use.

In another embodiment of the analysis system for evaluating physiological tissue state 1 according to the present invention, the control unit 14 further comprises a setting module (not shown). This setting module allows the user to set the desired state analysis mode. The analysis mode comprises: analyzing the amplitude of the waveform of the feedback wave against the comparison parameter, or analyzing the frequency peak value of the frequency domain information of the feedback wave against the comparison parameter. A single analysis mode can be adopted. For more advanced applications, both the waveform amplitude of the feedback wave and the frequency peak value of the frequency domain information of the feedback wave can be analyzed with the comparison parameter separately. This can further improve the accuracy of detection.

In summary, the analysis system for evaluating physiological tissue state of the present invention overcomes the limitations of traditional ultrasound imaging devices that cannot detect substantial changes in the lungs covered by air, water, or bone. It utilizes waves for detection, analyzing the reflected waveform amplitude or the characteristic frequency in the frequency domain information of the multiple wave segments to determine the state of the physiological tissue. Compared to existing detection tools such as ultrasound, which use a single frequency signal, this approach is more efficient and provides more interpretable results through the detected waveform amplitude or wave characteristic frequency, making it more user-friendly for the general public. Furthermore, because the analysis system for evaluating physiological tissue state can establish the characteristic model using the analyzed characteristic parameter, it can not only distinguish between normal and pathological parameters but also store additional data such as the subject's gender, age, weight, and height along with the analyzed comparison parameters to build a data parameter library for future artificial intelligence computations.

The examples above are only illustrative to explain principles and effects of the invention, but not to limit the invention. It will be apparent to those skilled in the art that modifications and variations can be made without departing from the spirit and scope of the invention. Therefore, the protection range of the rights of the invention should be as defined by the appended claims.

What is claimed is:

1. An analysis system for evaluating physiological tissue state, comprising:
a first fixing member, attached to a covering surface of a physiological tissue to be evaluated;
a second fixing member, installed at a predetermined interval from the first fixing member and attached to the covering surface of the physiological tissue;
a wave emitting unit, installed on the first fixing member and used to generate a detection wave for the physiological tissue;
a wave receiving unit, installed on the second fixing member and used to receive a feedback wave, and the feedback wave is the reflection of the detection wave transmitted through the physiological tissue as the transmission medium;
a control unit, electrically connected to the wave emitting unit and wave receiving unit, used to control the wave emitting unit to output the detection wave at a predetermined time interval, and correspondingly record the feedback wave received by the wave receiving unit; and
an analysis unit, electrically connected to the control unit, used to store multiple audio signals and comparison parameters, enabling the control unit to control the wave emitting unit to dynamically adjust the detection wave output by the wave emitting unit based on the multiple audio signals, and the analysis unit analyzes the feedback wave recorded by the control unit and the comparison parameters to obtain a characteristic parameter, thereby establishing a characteristic model for the physiological tissue.

2. The analysis system for evaluating physiological tissue state of claim 1, wherein the control unit selects an audio signal from the multiple audio signals to control the wave emitting unit to output the detection wave based on the selected audio signal, and the control unit provides the feedback wave received by the wave receiving unit to the analysis unit, enabling the analysis unit analyzes the waveform amplitude of the feedback wave against the comparison parameters to determine whether the physiological tissue is in a pathological state or a normal state, and the analysis unit then sets the waveform determined to be in the pathological state as a characteristic parameter and stores the characteristic parameter in the characteristic model.

3. The analysis system for evaluating physiological tissue state of claim 2, wherein when the analysis unit analyzes the waveform amplitude of the feedback wave against the comparison parameters, the control unit selects multiple audio signals from the multiple audio signals to control the wave emitting unit to output the detection wave based on the selected multiple audio signals, and the control unit provides the feedback wave received by the wave receiving unit to the analysis unit, allowing the analysis unit processes the time-domain information of the feedback wave using Fourier transform to obtain the frequency domain information of the feedback wave, and the analysis unit analyzes the frequency peaks in the frequency domain information against the comparison parameters to determine whether the physiological tissue is in the pathological state or the normal state, and the analysis unit then sets the waveform determined to be in the pathological state as the characteristic parameter and stores the characteristic parameter in the characteristic model.

4. The analysis system for evaluating physiological tissue state of claim 3, wherein the control unit is further equipped with a setting module that allows the user to set the desired state analysis mode, and the analysis mode comprises: analyzing the waveform amplitude of the feedback wave against the comparison parameters, analyzing the frequency peaks of the frequency domain information of the feedback wave against the comparison parameters, or jointly analyzing both the waveform amplitude of the feedback wave and the frequency peaks of the frequency domain information of the feedback wave.

5. The analysis system for evaluating physiological tissue state of claim 1, wherein the control unit controls the wave emitting unit to output the detection wave based on multiple audio signals from the multiple audio signals, and the control unit provides the feedback wave received by the wave receiving unit to the analysis unit, allowing the analysis unit processes the time-domain information of the feedback wave using Fourier transform to obtain the frequency domain information of the feedback wave, and analyzes the frequency peaks in the frequency domain information against the comparison parameters to determine whether the physiological tissue is in the pathological state or the normal state, and the analysis unit then sets the waveform determined to be in the pathological state as the characteristic parameter and stores the characteristic parameter in the characteristic model.

6. The analysis system for evaluating physiological tissue state of claim 5, wherein the control unit selects an audio signal from the multiple audio signals, and the control unit provides the feedback wave received by the wave receiving unit to the analysis unit, and the analysis unit analyzes the waveform amplitude of the feedback wave against the comparison parameters to determine whether the physiological tissue is in the pathological state or the normal state, and sets the waveform determined to be in the pathological state as the characteristic parameter and stores the characteristic parameter in the characteristic model.

7. The analysis system for evaluating physiological tissue state of claim 6, wherein the control unit is further equipped with a setting module that allows the user to set the desired analysis mode, and the analysis mode comprises analyzing the waveform amplitude of the feedback wave against the comparison parameters, analyzing the frequency peaks of the frequency domain information of the feedback wave against the comparison parameters, or jointly analyzing both the waveform amplitude of the feedback wave and the frequency peaks of the frequency domain information of the feedback wave.

8. The analysis system for evaluating physiological tissue state of claim 3, wherein the multiple audio signals have either increasing or decreasing wave frequencies.

9. The analysis system for evaluating physiological tissue state of claim 5, wherein the multiple audio signals have either increasing or decreasing wave frequencies.

10. The analysis system for evaluating physiological tissue state of claim 1, wherein the first fixing member and the second fixing member are patches.

11. The analysis system for evaluating physiological tissue state of claim 1, wherein the control unit and the analysis unit are connected via wired or wireless connection, and the analysis unit further stores a basic information of the evaluated subject, including gender, age, weight, and height, and the analysis unit establishes the characteristic parameters of the physiological tissue of the assessed obtained from the analysis and the basic information of the assessed to be data parameters required for artificial intelligence algorithms.
